(19) United States
(12) Patent Application Publication
(10) Pub. No.: US 2024/0142440 A1
(43) Pub. Date: May 2, 2024

(54) PHOTOSENSITIVE PROBES FOR TAGGING BIOMOLECULES

(71) Applicant: Syncell (Taiwan) Inc., Taipei City (TW)

(72) Inventors: Hsiao-Jen CHANG, New Taipei City (TW); Jung-Chi LIAO, Taipei City (TW)

(21) Appl. No.: 18/487,820

(22) Filed: Oct. 16, 2023

Related U.S. Application Data
(60) Provisional application No. 63/379,607, filed on Oct. 14, 2022.

(51) Int. Cl. G01N 33/532; C07D 307/91; G01N 33/58

(52) U.S. Cl. CPC G01N 33/532; C07D 307/91; G01N 33/58

(57) ABSTRACT

This disclosure provides photosensitive probes useful for photoactivated and tagging of subsets of biomolecules. These photosensitive probes described herein may be especially useful for selectively tagging and proximity labeling of biomolecules via selective light illumination through a microscope system. The methods and compositions may be particularly useful for analyzing biological samples, such as identifying proximal biomolecules in cell or tissue samples.

### CROSS REFERENCE OF RELATED APPLICATION

This application claims priority to U.S. Provisional Patent Application No. 63/379,607, filed on Oct. 14, 2022, titled “PHOTOSENSITIVE PROBES FOR TAGGING BIOMOLECULES,” which is herein incorporated by reference in its entirety.

### FIELD

The present invention relates to photosensitive probes useful for photoactivated and tagging of subsets of biomolecules, especially relating to analyze biological samples and identify proximal biomolecules in cell or tissue samples.

### BACKGROUND

Cells are composed of diverse types of biological molecules (biomolecules). The biomolecules in the cells interact with neighbor biomolecules in the subcellular environment to form complexes, organelles, or other assemblies and to carry out various cell functions. Characterizing the subcellular environment, within which biomolecules interact with one another, and how the biomolecules function together is very challenging. Biomolecules are small, and they exist in a cell environment with tens of millions of other molecules. The interactions between neighboring biomolecules are frequently weak, and techniques used to study biomolecules disrupt their interactions. While techniques such as yeast two-hybridization assays and more recently proximity labeling have advanced our understanding of the cell environment, these techniques suffer from various limitations such as nonspecific binding, slow reaction times and disruption of the natural cell environment, resulting in false positives and missed interactions. Specifically, the yeast two- hybridization assays have problems such as complicated operation, many false positives and false negatives, the results are mainly qualitative data, and it is not easy to accurately judge the strength of protein interactions; recent neighbor labeling techniques have problems such as slower reaction kinetics, lower temporal resolution (e.g., the BioID Proximity Labeling), and labeling specificity decreases when labeling time is too long (e.g., the TurboID Proximity Labeling, miniTurbo Proximity Labeling, TurboID Proximity Labeling). Therefore, what is needed are better tools for determining naturally occurring biomolecule interactions.

### SUMMARY OF THE DISCLOSURE

One aspect of the disclosure provides a photosensitive probe of formula (I):

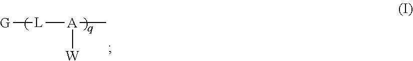

wherein the L portion includes a chemical bond or a linker; the G portion includes a conjugatable group for linking to a bait molecule, or a bait molecule; the A portion includes a triggerable molecule configured to render available a functional group linking the W portion thereof upon photo-uncaging; the W portion includes a caging group that cages the functional group of the triggerable molecule; and q is an integer of 1-20.

In this and other embodiments of a photosensitive probe, wherein the probe is bound to a bait molecule; the bait molecule is used for conjugation with a biological sample.

In any of these or other embodiments of the photosensitive probe, the photo-uncaging includes bond cleavage between the A portion and the W portion and removal of the W portion from the probe.

In any of these or other embodiments of the photosensitive probe, the bait molecule is one or more of an antibody, a CLIP-tag, a HaloTag, protein A, protein G, protein L, protein A/G, protein A/G/L, immunoglobulin binding peptides, avidin, streptavidin, neutravidin, an RNA molecule, a small molecule, a nucleic acid molecule, a fluorescent in situ hybridization (FISH) probe, fragment antigen binding region, nanobody, and a SNAP-tag.

In any of these or other embodiments of the photosensitive probe, the bait molecule is a secondary antibody.

In any of these or other embodiments of the photosensitive probe, the photosensitive probe satisfies one or more of the following conditions (1) to (5):(1) the functional group is a thiol group; (2) the caging group is a one- or/and two-photon sensitive caging group; (3) the linker includes a moiety of (PEG)ₙ, peptide, amino acid, oligonucleotide or a combination thereof, and wherein n is an integer of 1-20; (4) the conjugatable group is a click chemical group, —COOH, —NHS, a maleimide group, an iodoacetyl group or a cysteine/thiol group; (5) the caging group is removable at a wavelength ranging from 700 nm to 1600 nm at one- or/and two-photon light source so as to render available the functional group of the triggerable molecule.

In any of these or other embodiments of the photosensitive probe, the triggerable molecule is a cysteine or a derivative thereof with the thiol group thereof caged by the W portion.

In any of these or other embodiments of the photosensitive probe, the caging group is a nitrodibenzofuran-based caging group or an ortho-nitrobenzyl based caging group.

In any of these or other embodiments of the photosensitive probe, the caging group includes a moiety of

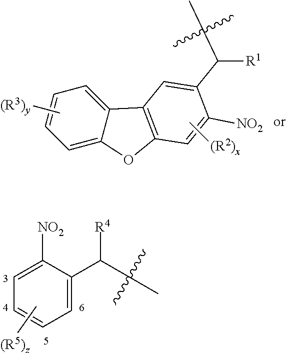

wherein R¹ is optionally substituted alkyl, optionally substituted carbocyclyl, optionally substituted heterocyclyl, optionally substituted aryl, optionally substituted heteroaryl, —OR^{D1} or —N(R^{D1a})₂; R² each independently is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D2} or —N(R^{D2a})₂; R₃ each independently is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D2} or —N(R^{D2a})₂; R⁴ is H, —CO₂R^{D1}, —C(═O)R^{D1} or —C(═O)N(R^{D1a})₂;
R⁵ each independently is halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D2} or —N(R^{D2a})₂; R^{D1} is hydrogen, optionally substituted alkyl, optionally substituted carbocyclyl, optionally substituted heterocyclyl, optionally substituted aryl, optionally substituted heteroaryl, or an oxygen protecting group; R^{D2} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group; R^{D1a} each independently is hydrogen, optionally substituted alkyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring; R^{D2a} each independently is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D2a} are taken together with their intervening atoms to form a substituted or unsubstituted heterocyclic or substituted or unsubstituted heteroaryl ring; x is 0, 1 or 2; y is 0, 1, 2, 3, or 4; and z is 0, 1, 2, 3, or 4.

In any of these or other embodiments of the photosensitive probe, R¹ is —OH, x is 0 and y is 0; and wherein R⁴ is H, R⁵ is C₁₋₂₀ alkoxy and z is 2, with the proviso that two R⁵ are at C-4 and C-5 positions, respectively.

In any of these or other embodiments of the photosensitive probe, the click chemical group is one or more of BCN, DBCO, N₃, and alkynyl.

In any of these or other embodiments of the photosensitive probe, the caging group is removable at a wavelength ranging from 200 nm to 1600 nm at one- or/and two-photon light source so as to render available the functional group of the triggerable molecule.

Another aspect of the disclosure provides a photoreactive kit, including: any of the above or other embodiments of the photosensitive probe; and a tag-bearing reactive molecule or an enzyme-bearing reactive molecule, having reactivity with the rendered available functional group of the triggerable molecule when it is uncaged.

In any of these or other embodiments of the photoreactive kit, the tag-bearing reactive molecule includes a tag moiety and a reactive moiety; and/or the enzyme-bearing reactive molecule includes an enzymatic moiety and a reactive moiety.

In any of these or other embodiments of the photoreactive kit, the tag moiety of the tag-bearing reactive molecule is at least one of a biotin derivative, a CLIP-tag, a digoxigenin tag, a HaloTag, a peptide tag, oligonucleotide, a SNAP-tag and a click chemistry tag; and/or
the enzymatic moiety is a peroxidase or a ligase.

In any of these or other embodiments of the photoreactive kit, the biotin derivative includes the moiety of

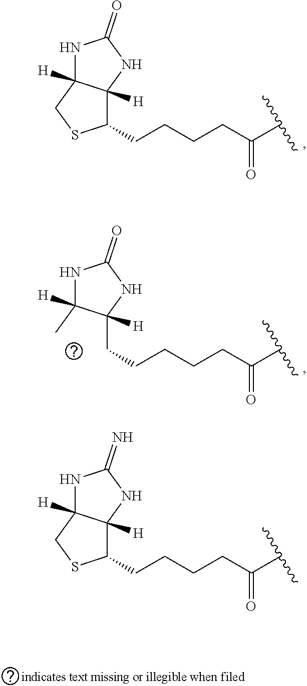

or a derivative thereof; and/or
the click chemistry tag includes an alkyne-based or azide-based moiety.

In any of these or other embodiments of the photoreactive kit, the click chemistry tag includes the moiety of

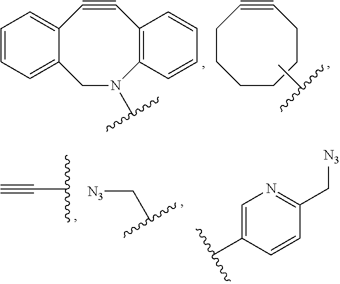

or a derivative thereof.

In any of these or other embodiments of the photoreactive kit, the reactive moiety of the tag-bearing reactive molecule includes a thiol-reactive moiety; and/or the reactive moiety of the enzyme-bearing reactive molecule includes a thiol-reactive moiety.

In any of these or other embodiments of the photoreactive kit, the reactive moiety of the tag-bearing reactive molecule includes an iodoacetyl-based moiety, maleimide-based moiety, pyridyldithio-based moiety, or HPDP-based moiety.

In any of these or other embodiments of the photoreactive kit, the tag-bearing reactive molecule is an iodoacetyl-(PEG)ₘ-biotin, maleimide-(PEG)ₘ-biotin, pyridyldithio-biotin or HPDP-biotin, and wherein m each independently is an integer of 0-20.

In any of these or other embodiments of the photoreactive kit, the kit further includes a connector conjugatable with the tag-bearing reactive molecule.

In any of these or other embodiments of the photoreactive kit, the kit further includes a tag-enzyme conjugatable with the connector.

In any of these or other embodiments of the photoreactive kit, the kit further includes a connector conjugatable with the tag-bearing reactive molecule, a tag-enzyme conjugatable with the connector, and a subject probe capable of forming a covalent bond with the biological sample by catalytic activity of the tag-enzyme; and/or further includes a subject probe configured to form a covalent bond with the biological sample by catalytic activity of the enzyme-bearing reactive molecule.

In any of these or other embodiments of the photoreactive kit, the tag-enzyme is a tag-peroxidase or a tag-ligase.

In any of these or other embodiments of the photoreactive kit, the subject probe includes a tag portion and a subject moiety.

In any of these or other embodiments of the photoreactive kit, a concentration of the photosensitive probe ranges from 0.1 μg/mL to 100 μg/mL and a concentration of the tag-bearing reactive molecule or the enzyme-bearing reactive molecule ranges from 1 μM to 20 mM.

Another aspect of the disclosure provides a method for photoreactive labeling, the method including the steps of: delivering the photosensitive probe of the photoreactive kit as described above or in another embodiment of the kit to a biological sample; conjugating the bait molecule to a target molecule in the biological sample; selectively illuminating a selected region of interest of the biological sample with optical radiation to uncage the photosensitive probe and thereby to generate an uncaged probe in the selected region of interest; and delivering the tag-bearing reactive molecule or the enzyme-bearing reactive molecule of the of the photoreactive kit as described above or in another embodiment of the kit to the biological sample so as to allow the tag-bearing reactive molecule or the enzyme-bearing reactive molecule to react with the uncaged probe in the selected region of interest.

Any of these or other embodiments of a method for photoreactive labeling can further include a step of removing the unconjugated photosensitive probe from the biological sample.

Any of these or other embodiments of a method for photoreactive labeling can further include a step of delivering the connector of the photoreactive kit as described above or in any other embodiments to the biological sample and conjugating the connector to the tag-bearing reactive molecule through the affinity between the connector and the tag-bearing reactive molecule.

Any of these or other embodiments of a method for photoreactive labeling can further include a step of delivering the tag-enzyme of the photoreactive kit as described above or in any other embodiments to the biological sample and conjugating the tag-enzyme to the connector so as to allow the tag-enzyme to catalyze the subject probe to form a covalent bond between the subject probe and the biological sample.

In any of these or other embodiments of a method for photoreactive labeling, the tag-enzyme activates the subject probe to form the covalent bond with a tyrosine of the biological sample.

Any of these or other embodiments of a method for photoreactive labeling can further include a step of delivering the subject probe of the photoreactive kit as described above or in any other embodiment so as to allow the enzyme-bearing reactive molecule to catalyze the subject probe to form a covalent bond between the subject probe and the biological sample.

In any of these or other embodiments of a method for photoreactive labeling, the enzyme-bearing reactive molecule activates the subject probe to form the covalent bond with a tyrosine of the biological sample.

In one embodiment, the method further comprises a step of removing the unconjugated photosensitive probe from the biological sample.

In one embodiment, the method further comprises a step of delivering the connector of the photoreactive kit to the biological sample and conjugating the connector to the tag-bearing reactive molecule through the affinity between the connector and the tag-bearing reactive molecule.

In one embodiment, the method further comprises a step of delivering the tag-enzyme of the photoreactive kit to the biological sample and conjugating the tag-enzyme to the connector so as to allow the tag-enzyme to catalyze the subject probe to form a covalent bond between the subject probe and the biological sample. The tag-enzyme activates the subject probe to form the covalent bond with a tyrosine of the biological sample.

In one embodiment, the method further comprises a step of delivering the subject probe of the photoreactive kit so as to allow the enzyme-bearing reactive molecule to catalyze the subject probe to form a covalent bond between the subject probe and the biological sample. The enzyme-bearing reactive molecule activates the subject probe to form the covalent bond with a tyrosine of the biological sample.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows a schematic depiction of a system useful for photoselective spatial tagging and proximity labeling in cells on a substrate.

FIG. 2A shows a schematic illustration of a photosensitive probe with a caging group.

FIG. 2B shows a schematic illustration of another photosensitive probe with a caging group.

FIG. 2C schematically illustrates a photo-labeling process for labeling biomolecules using the probe illustrated in FIG. 2A.

FIG. 3A shows a schematic illustration of a process (I) of direct photochemical labeling.

FIG. 3B shows a schematic illustration of a process (II) of photo-assisted enzymatic proximity labeling using the probe illustrated in FIG. 2A to label biomolecules in small region of interest (ROI).

FIG. 3C shows a schematic illustration of a process (III) of photo-assisted enzymatic proximity labeling similar to that shown in FIG. 3B with a signal amplification step.

FIG. 4 shows photolysis of the photosensitive probe illustrated in FIG. 2A caged by NDBF.

FIG. 5 shows photolysis of the photosensitive probes illustrated in FIG. 2A caged by DMNB.

FIGS. 6A-6K show examples of tag moieties that can be used in the tag-bearing reactive molecules described herein.

### DETAILED DESCRIPTION

The embodiments of the invention will be apparent from the following detailed description, which proceeds with reference to the accompanying drawings, wherein the same references relate to the same elements.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

As used in the description herein and throughout the claims that follow, the meaning of “a”, “an”, and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the content indicates the contrary. The recitation of ranges of values herein is merely intended to serve as a shorthand method of referring individually to each separate value falling within the range.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

### Abbreviations and Definitions

The term “antibody” refers to immunoglobulin and related molecules and includes monoclonal antibodies, polyclonal antibodies, monomers, dimers, multimers, multispecific antibodies (e.g., bispecific antibodies), heavy chain only antibodies, three chain antibodies, single chain Fv, nanobodies, etc., and also includes antibody fragments. An antibody may be a polyclonal or monoclonal or recombinant antibody. Antibodies may be murine, human, humanized, chimeric, or derived from other species. As used herein, when an antibody or other entity “specifically recognizes” or “specifically binds” an antigen or epitope, it preferentially recognizes the antigen in a complex mixture of proteins and/or macromolecules and binds the antigen or epitope with affinity, which is substantially higher than to other entities not displaying the antigen or epitope.

The term “bait molecule” refers to a molecule that specifically interacts with a molecule of interest, which may be referred to as a target (or prey). Examples of bait molecules include an antibody, CLIP-tag, a drug, a nucleic acid, a fluorescent in situ hybridization (FISH) probe, protein A, protein G, protein L, protein A/G, protein A/G/L, another small molecule, and a SNAP-tag.

The term “binding” refers to a first moiety physically interacting with a second moiety, wherein the first and second moieties are in physical contact with one another.

The term “biotin derivative” refers to a biotin moiety, including biotin and variations of biotin, such as biotin with an open ring or substitutions. Typically, a biotin derivative is easily detectable with a biotin-binding entity or protein, such as avidin, NeutrAvidin, or streptavidin.

The term “caging group” refers to a chemical group that functionally encapsulates part or all of a substance of interest in an inactive form. A caging group is typically part of a light sensitive probe and activates a triggerable portion of the probe under the action of energy, typically light.

The term “click chemistry” refers to a chemical approach that easily joins molecular building blocks. Typically, click chemistry reactions are efficient, high-yielding, reliable, create few or no byproducts, and are compatible with an aqueous environment or without an added solvent. An example of click chemistry is cycloaddition, such as the copper(I)-catalyzed [3+2]-Huisgen 1,3-dipolar cycloaddition of an alkyne and azide leading to the formation of 1,2,3-triazole or Diels-Adler reaction. Click chemistry also includes copper free reactions, such as a variant using substituted cyclooctyne (see e.g., J. M. Baskin et al., Proc. Natl. Acad. Sci. U.S.A. 2007 Oct. 23, 104 (43), 16793-16797). Other examples of click chemistry are nucleophilic substitutions; additions to C—C multiple bonds (e.g., Michael addition, epoxidation, dihydroxylation, aziridination); and nonaldol like chemistry (e.g., N-hydroxysuccinimide active ester couplings). Click chemistry reactions can be bioorthogonal reactions, but do not need to be.

The term “conjugate” refers to a process by which two or more molecules specifically interact. In some embodiments, a tag and a label conjugate. In some embodiments, a catalyzed reporter deposition and a biomolecule conjugate.

The term “conjugatable” refers to a molecule that can specifically come together with another molecule to which it can be conjugated. In some embodiments, a bait is conjugatable to a biomolecule of interest. In some embodiments, a connector is conjugatable to a primary subject probe.

The term “detectable label” refers to a compound or composition which is or is configured to be conjugated directly or indirectly to a molecule. The label itself may be detectable and be a directly detectable label (such as, e.g., fluorescent labels such as fluorescent chemical adducts, radioisotope labels, etc.), or the label can be indirectly detectable (such as, e.g., in the case of an enzymatic detectable label, the enzyme may catalyze a chemical alteration of a substrate compound or composition and the product of the reaction is detectable). Examples of detectable labels include e.g., a biotin label, a fluorescent label, horseradish peroxidase, an immunologically detectable label (e.g., a hemagglutinin (HA) tag, a poly-histidine tag), another light emitting label, and a radioactive label. An example of an indirect label is biotin, which can be detected using a streptavidin detection method.

The term “immunoglobulin-binding protein” refers to immunoglobulin-binding bacterial proteins and variations of immunoglobulin-binding bacterial proteins. Examples include protein A, protein G, protein L, protein A/G, and protein A/G/L. Protein A and protein G and are bacterial proteins originally obtained from Staphylococcus aureus and Group G Streptococci, respectively, and have high affinity for the Fc region of IgG type antibodies. Protein A/G combines the binding domains of protein A and protein G. Protein A/G/L combines binding domains of protein A, protein G, and protein L. Immunoglobulin-binding proteins bind to specific domain of antibodies.

The term “instructional material” includes a publication, a recording, a diagram, a link, or any other medium of expression which can be used to communicate the usefulness of one or more compositions of the disclosure for its designated use. The instructional material of a kit may, for example, be affixed to a container which contains the composition or components or be shipped together with a container which contains the composition or components. Alternatively, the instructional material may be shipped separately from a container with the intention that the instructional material and a composition or component be used cooperatively by the recipient.

The term “label” refers to a molecule which produces or can be induced to produce a detectable signal. In some embodiments, a label produces a signal for detecting a neighboring biomolecule. Examples of labels that can be used include avidin labels, NeutrAvidin labels, streptavidin labels to detect a biotin tag.

The term “linker” refers to a structure which connects two or more substructures. A linker has at least one uninterrupted chain of atoms extending between the substructures. The atoms of a linker are connected by chemical bonds, typically covalent bonds.

The phrases “bound to”, “coupled to”, “conjugated to”, “conjugatable to”, “attached to” and “linked to” refer to being directly or indirectly bound/conjugated/attached/linked. For instance, a bait molecule can be directly attached to a triggerable molecule without intervening atoms, groups, or moieties therebetween. Alternatively, a bait molecule may be indirectly attached to the triggerable molecule by one or more intervening atoms, groups, moieties, or linkers therebetween. The intervening atoms, groups, moieties, or linkers may include, for example, one or more non-carbon atoms, groups, or moieties, or an unsubstituted or substituted alkylene or alkenylene group which may include amine, amide, ether, ester, or thioester linkages, and optionally be interrupted by one or more heteroatoms and/or rings, including aromatic rings optionally substituted.

The term “photoactivated” or “light activated” refers to excitation of atoms by means of radiant energy (e.g., by a specific wavelength or wavelength range of light, UV light, etc.). In some examples, a photoactivated probe has a free thiol group and can react with a thiol-reactive molecule.

The term “photo-uncaging” or “uncaging” refers to a process of removing caging groups by means of radiant energy (e.g., by a specific wavelength or wavelength range of light, UV light, etc.). In some examples, NDBF-caged probes can be deprotected by photoremoval of NDBF groups to yield photo-uncaged probes.

The term “proximity molecule” or neighboring molecule refers to a molecule that is near another molecule. A proximity molecule or neighbor molecule may be bound to the molecule (e.g., covalently or non-covalently) or may be close by and not bound to the molecule.

The term “prey” refers to a binding partner of a bait molecule. For example, if an antibody is a bait, a corresponding protein to which the bait molecule can bind is the corresponding prey. In some embodiments, a bait can bind with a single prey. In some embodiments, a bait can bind with more than one prey.

The term “protein tag” refers to peptide sequences of amino acids with a tag. Protein tags are typically conjugated to a label. An example of a protein tag is a “self-labeling” protein tag configured to covalently bind to an appropriate label or ligand with a cognate binding region. The binding reaction between a self-labeling protein tag and label or ligand typically has rapid labeling kinetics, low substrate promiscuity, and thermodynamic stability. Examples of self-labeling tags include BL-Tag, CLIP-tag, covalent TMP tag, Halotag, and SNAP-tag. SNAP-tag is a ˜20 kDa variant of the DNA repair protein O6-alkylguanine-DNA alkyltransferase that specifically recognizes and rapidly reacts with benzylguanine (BG) derivatives. During a labeling reaction, the benzyl moiety of the benzylguanine is covalently attached to the SNAP-tag, and guanine is released. CLIP-tag is a variation of SNAP-tag configured to react specifically with O2-benzylcytosine (BC) derivatives rather than benzylguanine (BG).

The term “secondary antibody” refers to an antibody that specifically recognizes a region of another antibody. A secondary antibody generally recognizes the Fc region of a particular isotype of antibody. A secondary antibody may recognize the Fc region from one or more particular species.

The term “small molecule” refers to low molecular weight molecules that include carbohydrates, drugs, enzyme inhibitors, lipids, metabolites, monosaccharides, natural products, nucleic acids, peptides, peptidomimetics, second messengers, small organic molecules, and xenobiotics. Typically, small molecules are less than about 1000 molecular weight or less than about 500 molecular weight.

The term “tag” refers to a functional group, compound, molecule, substituent, or the like, that can enable detection of a target molecule. A tag can enable a detectable biological or physiochemical signal that allows detection via any means, e.g., absorbance, chemiluminescence, colorimetry, fluorescence, luminescence, magnetic resonance, phosphorescence, radioactivity. The detectable signal provided due to the tag can be directly detectable due to a biochemical or physiochemical property of the tag moiety (e.g., a fluorophore tag) or indirectly due to the tag interaction with another compound or agent. Typically, a tag is a small functional group or small organic compound. In some embodiments, the employed tag has a molecular weight of less than about 1,000 Da, less than 750 Da, less than 500 Da or even smaller.

The term “tagging” refers to the process of adding a tag to a functional group, compound, molecule, substituent, or the like. Typically, tagging enables detection of a target molecule.

The term “triggerable portion” refers to a molecule or a portion of a molecule having a masked moiety, which, upon exposure to energy (e.g., a specific wavelength of light or wavelength range of light, UV light, a chemical) has a change in reactivity or activity. Typically, the change involves a chemical change, such that bonds are broken. For example, upon exposure to light, a cysteine molecule with a thiol group masked by NDBF or DMNB releases a thiol group and the remaining molecule is chemically reactive.

The term “tyramide signal amplification (TSA)”, refers to a catalyzed reporter deposition (CARD) an enzyme-mediated detection method that utilizes catalytic activity of an enzyme (e.g., horseradish peroxidase) to catalyze inactive tyramide to highly active tyramide. The amplification can take place in the presence of low concentrations of hydrogen peroxide (H₂O₂). In some examples, tyramide can be labeled with a detectable label, such as a fluorophore (such as biotin or 2,4-dinitrophenol (DNP)).

### Photosensitive Probes

This disclosure describes photosensitive probes that can label biomolecules and their neighboring biomolecules, while largely maintaining naturally occurring molecular structure in the biomolecules. The probes described herein may be particularly useful for specifically labeling subsets of biomolecules in subcellular regions of cells using an image guided microscope with precision illumination control such as the system described in U.S. Pat. No. 11,265,449B2, to enable automatic labeling of cellular biomolecules of interest. The probes can be used for in situ tagging of biomolecules such as proteins inside cells or tissues and that can be followed by proximity labeling such as using Tyramide Signal Amplification (TSA). The biomolecules can be further analyzed by analytical techniques such as mass spectrometry and sequencing. Therefore, these photosensitive probes may be especially useful for performing omics studies, such as genomics, proteomics, and transcriptomics, and for finding relevant biomarkers for diagnosis and treatment.

In one aspect, the photosensitive probes represents by the formula (I):

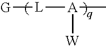

wherein the L portion includes a chemical bond or a linker, the G portion includes a conjugatable group for linking to a bait molecule, or a bait molecule, the W portion includes a caging group that cages the functional group of the triggerable molecule, the A portion includes a triggerable molecule configured to render available a functional group linking the W portion thereof upon photo-uncaging, and q is an integer of 1-20. It will be appreciated that q is within the range of values described above, and the greater the value of q, the greater the retrieved signal can be amplified. In one embodiment, the photo-uncaging comprises bond cleavage between the A portion and the W portion and removal of the W portion from the probe.

As described above, the photosensitive probes may be configured to have a bait molecule or a conjugatable group for linking to a bait molecule. Not-limiting examples of the conjugatable group for linkage with the bait molecule can include a click chemical group (such as BCN

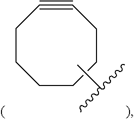

### DBCO

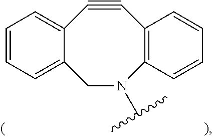 [SMILES: *N1Cc2ccccc2C#Cc2ccccc21]

### N3

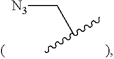 [SMILES: *CN=[N+]=[N-]]

alkynyl

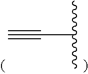 [SMILES: *C#C]

or the like), —COOH, —NHS, a maleimide group, an iodoacetyl group, a cysteine/thiol group and the like. Not-limiting examples of bait molecules can include an antibody, a CLIP-tag, HaloTag, a SNAP-tag, a functional protein (e.g. protein A, protein G, protein L, protein A/G, protein A/G/L, or a protein drug), immunoglobulin binding peptides, avidin, streptavidin, neutravidin, an RNA molecule, a small molecule (e.g. erlotinib), a nucleic acid molecule, a fluorescent in situ hybridization (FISH) probe, fragment antigen binding region, nanobody , a biologic drug, and the like. Examples of biologic drugs that can be used as bait moleculeinclude abatacept (Orencia); abciximab (ReoPro); abobotulinumtoxinA (Dysport); adalimumab (Humira); adalimumab-atto (Amjevita); ado-trastuzumab emtansine (Kadcyla); aflibercept (Eylea); agalsidase beta (Fabrazyme); albiglutide (Tanzeum); aldesleukin (Proleukin); alemtuzumab (Campath, Lemtrada); alglucosidase alfa (Myozyme, Lumizyme); alirocumab (Praluent); alteplase, cathflo activase (Activase); anakinra (Kineret); asfotase alfa (Strensiq); asparaginase (Elspar); asparaginase erwinia chrysanthemi (Erwinaze); atezolizumab (Tecentriq); basiliximab (Simulect); becaplermin (Regranex); belatacept (Nulojix); belimumab (Benlysta); bevacizumab (Avastin); bezlotoxumab (Zinplava); blinatumomab (Blincyto); brentuximab vedotin (Adcetris); canakinumab (Ilaris); capromab pendetide (ProstaScint); certolizumab pegol (Cimzia); cetuximab (Erbitux); collagenase (Santyl); collagenase clostridium histolyticum (Xiaflex); daclizumab (Zenapax); daclizumab (Zinbryta); daratumumab (Darzalex); darbepoetin alfa (Aranesp); denileukin diftitox (Ontak); denosumab (Prolia, Xgeva); dinutuximab (Unituxin); dornase alfa (Pulmozyme); dulaglutide (Trulicity); ecallantide (Kalbitor); eculizumab (Soliris); elosulfase alfa (Vimizim); elotuzumab (Empliciti); epoetin alfa (Epogen/Procrit); etanercept (Enbrel); etanercept-szzs (Erelzi); evolocumab (Repatha); filgrastim (Neupogen); filgrastim-sndz (Zarxio); follitropin alpha (Gonal f); galsulfase (Naglazyme); glucarpidase (Voraxaze); golimumab (Simponi); golimumab injection (Simponi Aria); ibritumomab tiuxetan (Zevalin); idarucizumab (Praxbind); idursulfase (Elaprase); incobotulinumtoxinA (Xeomin); infliximab (Remicade); infliximab-dyyb (Inflectra); interferon alfa-2b (Intron A); interferon alfa-n3 (Alferon N Injection); interferon beta-1a (Avonex, Rebif); interferon beta-1b (Betaseron, Extavia); interferon gamma-1b (Actimmune); ipilimumab (Yervoy); ixekizumab (Taltz); laronidase (Aldurazyme); mepolizumab (Nucala); methoxy polyethylene glycol-epoetin beta (Mircera); metreleptin (Myalept); natalizumab (Tysabri); necitumumab (Portrazza); nivolumab (Opdivo); obiltoxaximab (Anthim); obinutuzumab (Gazyva); ocriplasmin (Jetrea); ofatumumab (Arzerra); olaratumab (Lartruvo); omalizumab (Xolair); onabotulinumtoxinA (Botox); oprelvekin (Neumega); palifermin (Kepivance); palivizumab (Synagis); panitumumab (Vectibix); parathyroid hormone (Natpara); pegaspargase (Oncaspar); pegfilgrastim (Neulasta); peginterferon alfa-2a (Pegasys); peginterferon alfa-2b (PegIntron, Sylatron); peginterferon beta-1a (Plegridy); pegloticase (Krystexxa); pembrolizumab (Keytruda); pertuzumab (Perjeta); ramucirumab (Cyramza); ranibizumab (Lucentis); rasburicase (Elitek); raxibacumabreslizumab (Cinqair); reteplase (Retavase); rilonacept (Arcalyst); rimabotulinumtoxinB (Myobloc); rituximab (Rituxan); romiplostim (Nplate); sargramostim (Leukine); sebelipase alfa (Kanuma); secukinumab (Cosentyx); siltuximab (Sylvant); tbo-filgrastim (Granix); tenecteplase (TNKase); tocilizumab (Actemra); trastuzumab (Herceptin); ustekinumab (Stelara); vedolizumab (Entyvio); ziv-aflibercept (Zaltrap). As an alternative, the photosensitive probe may be configured to have a conjugatable group for linking to a bait molecule. The linker included in the L portion of the formula (I) can include the moiety of (PEG)ₙ (i.e.

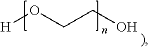

peptide, amino acid, oligonucleotide or a combination thereof, and wherein n each independently is an integer of 1-20. Other examples of polymeric linkers include polypropylene glycol, polyethylene, polypropylene, polyamides, and polyesters. The linker can be linear molecules in a chain of at least one or two atoms and can include more. In some embodiments, the “L-A” portion in the formula (I) may include a cysteine-containing peptide chain with cysteine caged by the W portion. Optionally, the cysteine-containing peptide chain may be elongated with one or more (PEG)ₙ block.

The caging group included in the W portion of the formula (I) can be any suitable photo-cleavable protecting group for functional group of the triggerable molecule. For example, one- and/or two-photon sensitive protecting group may be employed to cage the thiol functionality of cysteine. In some embodiments, the photosensitive probe may be configured to include a nitrodibenzofuran-based caging group or an ortho-nitrobenzyl based caging group as the caging group bonded to a sulfur atom of the triggerable molecule.

As an example of the two-photon sensitive probe, the moiety represented by formula (II) may be used for the W portion of the photosensitive probe:

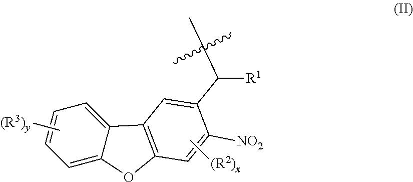

wherein, R¹ is optionally substituted alkyl (i.e. chain alkyl), optionally substituted carbocyclyl (i.e. alicyclyl), optionally substituted heterocyclyl, optionally substituted aryl, optionally substituted heteroaryl, —OR^{D1} or —N(R^{D1a})2; R² each independently is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D2} or —N(R^{D2a})₂; R³ each independently is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D2} or —N(R^{Da2})2; R^{D1} is hydrogen, optionally substituted alkyl, optionally substituted carbocyclyl, optionally substituted heterocyclyl, optionally substituted aryl, optionally substituted heteroaryl, or an oxygen protecting group; R^{D2} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group; R^{D1a} each independently is hydrogen, optionally substituted alkyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring; R^{D2a} each independently is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D2a} are taken together with their intervening atoms to form a substituted or unsubstituted heterocyclic or substituted or unsubstituted heteroaryl ring; x is 0, 1 or 2; and y is 0, 1, 2, 3, or 4. In a preferred implantation, the photosensitive probe includes nitrodibenzofuran (NDBF, i.e. for the formula (II), R1 being —OH, and x and y being 0) as an efficient thiol caging group that can undergo photocleavage to liberate a free thiol upon two-photon irradiation with, for example, 800 nm.

The above NDBF-caged probe also may be used as a one-photon sensitive probe, which can show uncaging properties upon one-photon irradiation with, for example, 365 nm. Alternatively, the moiety represented by formula (III) may be used for the W portion of the photosensitive probe with efficient one-photo sensitivity:

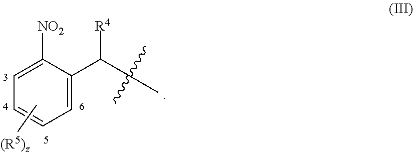

wherein, R⁴ is H, —CO₂R^{D1}, —C(═O)R^{D1} or —C(═O)N(R^{D1a})₂; R⁵ each independently is halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D2} or —N(R^{D2a})₂; R^{D1} is hydrogen, optionally substituted alkyl, optionally substituted carbocyclyl, optionally substituted heterocyclyl, optionally substituted aryl, optionally substituted heteroaryl, or an oxygen protecting group when attached to an oxygen atom; R^{D2} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group; R^{D1a} each independently is hydrogen, optionally substituted alkyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring; R^{D2a} each independently is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D2a} are taken together with their intervening atoms to form a substituted or unsubstituted heterocyclic or substituted or unsubstituted heteroaryl ring; and z is 0, 1, 2, 3, or 4. In a preferred implantation, the photosensitive probe includes 4,5-dimethoxy-2-nitrobenzyl (DMNB, i.e. for the formula (III), R⁴ being H, z being 2 and R⁵ being methoxy at C-4 and C-5 positions) for thiol protection through thioether bond, and can be photo-uncaged upon one-photon irradiation with, for example, 350 nm.

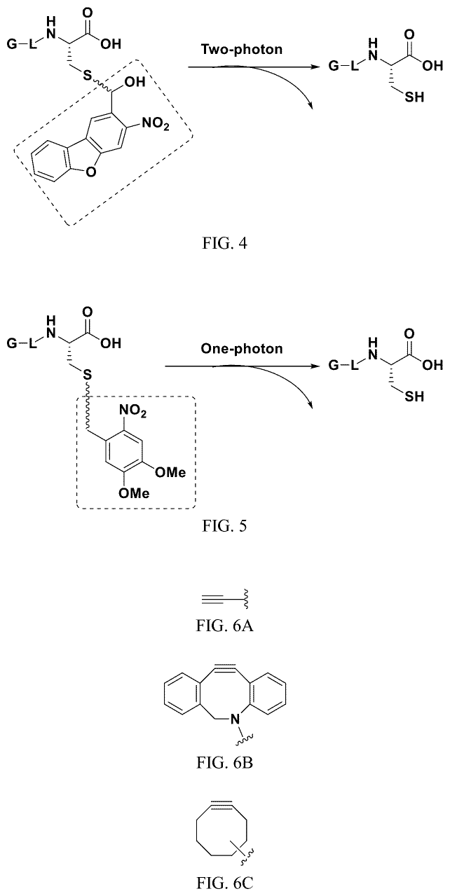

FIGS. 4 shows that photolysis of the photosensitive probes having a conjugatable group (labeled as G) on the N-terminal end of the peptide-based linker (labeled as L) and NDBF for masking the critical cysteine residue undergoes uncaging upon UV irradiation. Also, FIGS. 5 shows photo-uncaging of the photosensitive probes including DMNB-caged cysteine-containing peptide. The selection of a particular caging group can depend on the desired wavelength and the types of the bait molecule. For example, the constituents of the photosensitive probe and constituents for the pre-probe analysis can be chosen so as to not interfere (or minimally interfere) with each other. Additionally, the photosensitive probes described herein may further include different additional protecting groups for caging one or more functionalities in the peptide-based linker L. As a result, different functionalities can be selectively uncaged using different-wavelength irradiation.

Not-limiting examples of reactive moieties for the tag-bearing reactive molecules and the enzyme-bearing reactive molecules described herein can include maleimide-based moiety, iodoacetyl-based group moiety, pyridyldithio-based moiety, HPDP-based moiety or others having reactivity with the free functional group of the uncaged probe (e.g. any thiol-reactive moiety).

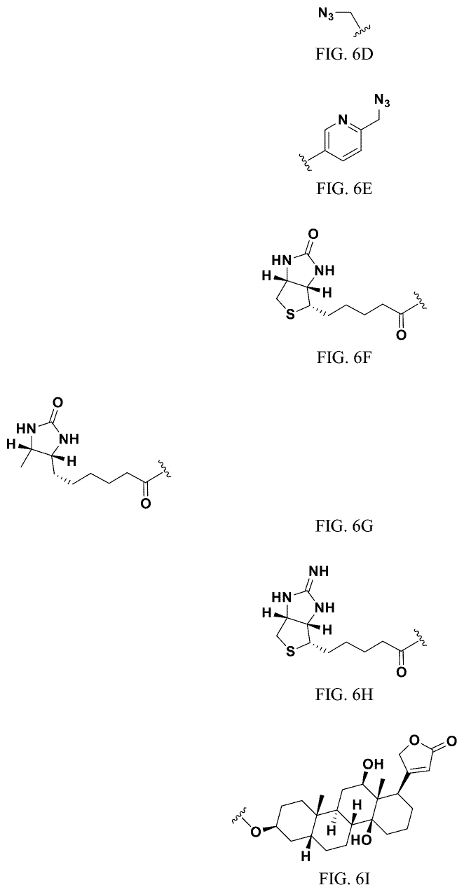

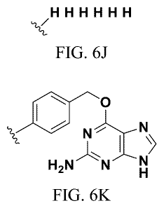

FIGS. 6A-6K show examples of tag moieties that can be used in the tag-bearing reactive molecules described herein. The tag portions are configured to interact with a detectable label to label biomolecules neighboring a target molecule of interest. FIGS. 6A-6E shows examples of click chemistry tags that can be used in the tag-bearing reactive molecules. The click chemistry tag may be, for example, an azide moiety or an alkyne moiety. FIGS. 6F-6H shows examples of biotin derivatives that can be used for the tag-bearing reactive molecules. FIG. 6I shows a digoxigenin moiety tag. FIG. 6J shows a peptide tag. In particular, FIG. 6J shows a poly His tag with 6 histidines. However, a histidine tag could instead fewer or more histidines, such as 5 or 7-10 or more. FIG. 6K shows a SNAP-tag. In some instances, a CLIP-tag or HaloTag could also be used.

The subject probe described herein can include a tag portion and a subject moiety. After activation, a reactive intermediate (such as a free radical) can be generated from the subject moiety and poised for covalent bond formation with amino acid (e.g. tyrosine) in proximity. Any tag described herein for the tag-bearing reactive molecule also can be used in the subject probe.

Photoselective tagging and labeling as described herein can be performed in various types of samples, such as samples obtained from tissues, cells, or particles, such as from an entity (e.g., a human subject, a mouse subject, a rat subject, an insect subject, a plant, a fungi, a microorganism, a virus) or tissues samples or cell samples that are not from an organism, such as cell culture samples or artificial tissue scaffold samples (e.g., cultured laboratory cells, in vitro developed heart tissue, 3-d printed tissue, etc.). Samples for analysis using the probes, materials, and methods described herein can be living (live cells) or can be not living (e.g., fixed). A sample for tagging and labeling can include a monolayer sample, a multi-layer sample, a sample fixed to a substrate (e.g., a microscope slide), a sample not fixed to a substrate, a suspension of cells, or an extract, such as an in vitro cell extract, a reconstituted cell extract, or a synthetic extract. In some embodiments, a sample is not fixed (unfixed). Examples of probes useful for tagging live cells include those utilizing a small molecule or those sometimes referred to as self-labeling molecules (e.g., Clip-tag, Halotag, SNAP-tag). In some embodiments, a large number of cells can be automatically analyzed using the methods and materials described herein (e.g., at least about 1,000 cells, at least 10,000 cells, at least 100,000 cells, at least 1 million cells). In some embodiments, a smaller number of cells can be analyzed, such as no more than 1,000 cells, no more than 100 cells, or only a few cells or a single cell. In some embodiments a sample is fixed. For example, a cell or tissue sample may be fixed with e.g., acetic acid, acetone, formaldehyde (4%), formalin (10%), methanol, glutaraldehyde, or picric acid. A fixative may be a relatively strong fixative and may crosslink molecules or may be weaker and not crosslink molecules. A cell or tissue sample for analysis may be frozen, such as using dry ice or flash frozen, prior to analysis. A cell or tissue sample may be embedded in a solid material or semi-solid material such as paraffin or resin prior to analysis. In some embodiments, a cell or tissue sample for analysis may be subject to fixation followed by embedding, such as formalin fixation and paraffin embedding (FFPE).

The concentration of the photosensitive probe can range from 0.1 μg/mL to 100 μg/mL, while the concentration of the tag-bearing reactive molecule or the enzyme-bearing reactive molecule can range from 1 μM to 20 mM. The wavelength of light for activation of the photosensitive probe or photoselective tagging and labeling ranges in some embodiments from about 200 nm to about 1600 nm. In some embodiments, the wavelength of light for performing photoselective tagging and labeling ranges from about 700 nm to about 1600 nm (e.g. 800 nm) at two-photon light source; or ranges from about 300 nm to about 650 nm (e.g. 365 nm) at single-photon light source. The wavelengths used for photoactivation of the probe are different from the wavelengths used for imaging. In some embodiments, the activation of the photosensitive probe utilizes optical radiation (light) at from around 300-450 nm, 550 nm for single photon activation or >720 nm for multiphoton activation. The particular wavelength depends on the particular caging group of the photosensitive probe.

### Kits

This disclosure also provides a photoreactive kit comprising aforementioned photoreactive probes, and a tag-bearing reactive molecule or an enzyme-bearing reactive molecule, having reactivity with the rendered available functional group of the triggerable molecule when it is uncaged. The photoreactive kits will typically include instructional materials disclosing means for generating or modifying the one or more probes, such as attaching a bait molecule to a triggerable molecule masked by a caging group to prepare a photosensitive probe, applying the photosensitive probe to a sample, conjugating the bait molecule of the photosensitive probe to a prey molecule (in the sample), removing (washing away) unconjugated photosensitive probe, photo-uncagging the photosensitive probe to generate an uncaged probe, applying a tag-bearing reactive molecule or an enzyme-bearing reactive molecule to the sample, reacting the tag-bearing reactive molecule or the enzyme-bearing reactive molecule with the uncaged probe, removing (washing away) unreacted tag-bearing reactive molecule or unreacted enzyme-bearing reactive molecule, and applying labels to the sample. The kits can optionally include instructional materials teaching the use of the photosensitive probes, the tag-bearing reactive molecules, the enzyme-bearing reactive molecules, the labels, and wash solution and the like.

The kits may also include additional components to facilitate the particular application for which the kit is designed. Thus, for example, the photoreactive kit may additionally contain means of detecting the sample and/or detecting the label (e.g., enzyme substrates for enzymatic labels, filter sets to detect fluorescent labels, enzymes or associated detection reagents, including reagents for performing catalyzed reporter deposition (CARD) or signal amplification (e.g., avidin, Neutravidin, streptavidin, HRP, tyramide, hydrogen peroxide, etc.). The photoreactive kits may additionally include wash solutions, such as blocking agents, detergents, salts (e.g., sodium chloride, potassium chloride, phosphate buffer saline (PBS)) for one or more steps (e.g., after sample fixation). The photoreactive kits may include variations of wash solutions, such as concentrates of wash buffers configured to be diluted before use or components to use for making one or more wash solutions) and other reagents routinely used for the practice of a particular method. The photoreactive kits may include fixatives and other sample preparation materials (e.g., ethanol, methanol, formalin, paraffin, etc.)

### Method for Photoreactive Labeling

Certain exemplary embodiments according to the present disclosure are described below.

FIG. 1 shows a schematic depiction of a system useful for photoselective spatial tagging and labeling. The bottom part of FIG. 1 shows substrate 106, such as a microscope stage, and a monolayer of plurality of cells 108 disposed on the substrate. In some embodiments, the surface of an entire substrate, or a portion of the substrate, can be analyzed using an automated microscope system to identify a region of interest. For example, a sample can be stained or labeled to identify a region of interest. The top part of FIG. 1 shows an expanded view of cell 108a, one of the plurality of cells 108. The cell 108a has a nucleus 116 and a plurality of different types of organelles 112, such as cell membranes, mitochondria, ribosomes, and vacuoles. Microscope system 102 selectively shines narrow band of light 104 onto region of interest (ROI) 118 for analysis of the region of interest 118. The illumination can be selective, and large regions 114 of the cell and substrate are not illuminated. As explained in more detail below, narrow band of light 104 activates a photosensitive probe in only the region of interest 118.

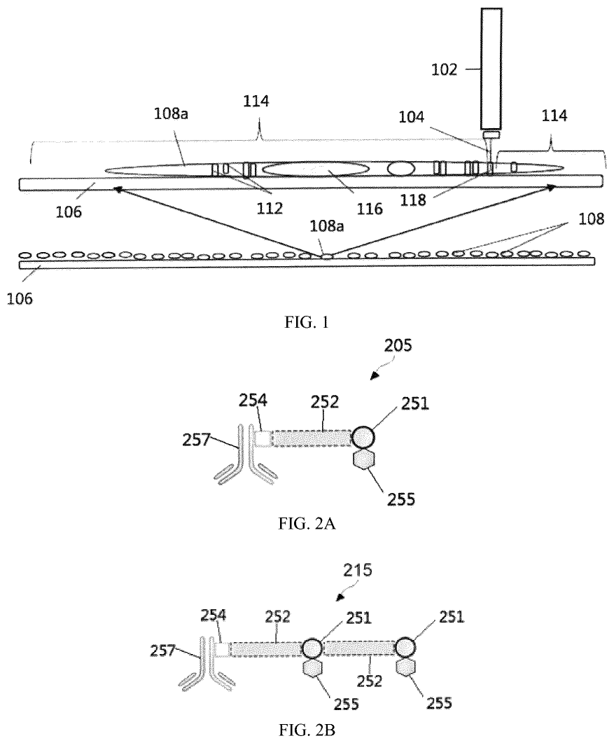

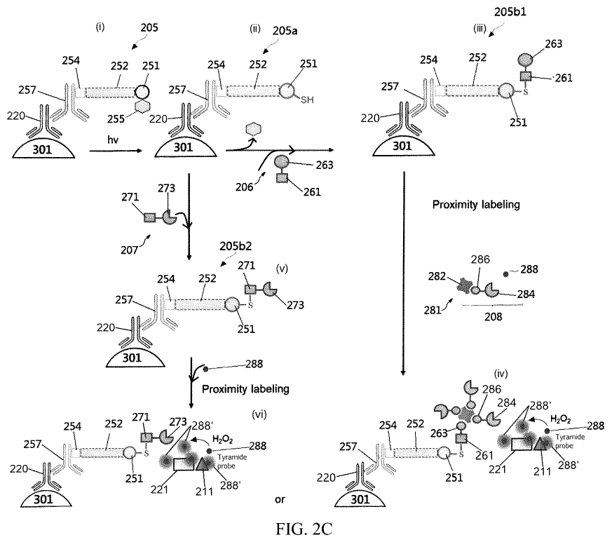

FIG. 2A schematically illustrates photosensitive probe 205 and FIG. 2B schematically illustrates another photosensitive probe 215. Photosensitive probe 205 and photosensitive probe 215 are similar except that photosensitive probe 215 includes two repeating units consisting of a triggerable portion 251, a linker 252 and caging group 255. (Photosensitive probe is interchangeably referred to herein as photoreactive and uncageable probe, photoreactive and deprotectable probe, photo-uncageable probe, photo-deprotectable probe, or probe unless specific context indicates otherwise). Photosensitive probe 205 has a triggerable portion 251 caged by caging group 255. FIG. 2A shows photosensitive probe 205 linked to a bait molecule 257. In this example, bait molecule 257 is an antibody and the triggerable portion 251 of the probe 205 is attached to the bait molecule 257 through a linker 252 bonded to the triggerable portion 251 and to a conjugatable group 254 conjugated to the bait molecule 257. In other examples, the linker 252 may be replaced with a chemical bond, and the triggerable portion 251 may be bonded to the bait molecule 257 without the linker 252 and the conjugatable group 254 therebetween. The triggerable portion 251 may be directly bound to bait molecule 257. Further, although this example shows the bait molecule 257 as an antibody (such as a secondary antibody), any bait molecule as described herein can be used.

FIG. 2C panel (i) shows target biomolecule 301, the target biomolecule to which neighbor molecules 211 can be identified using the methods herein. (Neighbor molecules 211 are present throughout this process, but for simplicity are only illustrated in FIG. 2C's panels (iv) and (vi)). The target biomolecule 301 is typically in a cell (cell 108a shown in FIG. 1) in a sample on a microscope slide, though does not need to be. Primary antibody 220 has recognized and bound to target biomolecule 301. Photosensitive probe 205 with bait molecule 257 (secondary antibody) has been delivered to the sample and detects and binds to target biomolecule 301 through recognizing and binding to primary antibody 220. As shown in FIG. 2C's panel (ii), after the photosensitive probe 205 and bait molecule 257 delivered to the sample detects and binds to target biomolecule 301, light (hv) is applied to the region and the photoreactive probe 205 is activated by the process of photo-uncaging, leading to removal of caging group 255 from photoreactive probe 205 and leaving behind on activated probe 205a a reactive entity (a —SH entity, in this case). In a first pathway, tag-bearing reactant 206 (also referred to interchangeably herein as tag-bearing reactive molecule unless specific context indicates otherwise) is added to the sample. Tag-bearing reactant 206 includes reactive moiety 261 and tag moiety 263. As illustrated in FIG. 2C's panel (iii), reactive moiety 261 of tag-bearing reactant 206 is reacting with activated probe 205a conjugated to the target biomolecule 301. Reactive moiety 261 (e.g., a thiol-reactive moiety) of the tag-bearing reactant 206 binds with the free functional group (e.g., thiol group) of the activated probe 205a to thus form a tag-bearing probe 205b1 conjugated to the target biomolecule 301. In this example, tag moiety may be, for example, iodoacetyl-(PEG)ₘ-biotin, maleimide-(PEG)ₘ-biotin, pyridyldithio-biotin, HPDP-biotin or the like (m being an integer of 0-20). Although this example shows photosensitive probe 205 binding to primary antibody 220, the photosensitive probe 205 can detect and bind to any target as described herein. Further, after linkage between the activated probe 205a and tag-bearing reactant 206, the tag moiety 263 of the tag-bearing reactant 206 can be detected using the methods described herein. For example, if the tag moiety 263 is a biotin derivative, it can be detected using an avidin/streptavidin/neutravidin detection method using one or more of avidin, streptavidin, and neutravidin. This method does not need to have a cleavable linker and need not rely upon a cleavage step or de-hybridization after photolabeling. This method may reduce the number of steps required and/or reduce complexity, cost, etc.

FIG. 2C also shows an example of a labeling system 208 that can be used with the tag-bearing probe 205b1 to label biomolecules neighboring a target biomolecule of interest. The labeling system 208 includes a labeling complex 281 with a connector 282 and an enzyme or catalyst 284, and a subject probe 288. In some embodiments, the connector 282 can be avidin-dye conjugate, streptavidin-dye conjugate, neutravidin-dye conjugate or the like, and the enzyme or catalyst 284 can be tag-peroxidase and utilizes peroxide (not shown) for activity. As seen in FIG. 2C's panel (iv), the tag moiety 263 of the tag-bearing reactant 206 and the connector 282 recognize one another and conjugate. The tag moiety 286 of the enzyme 284/tag moiety 286 entity also recognizes connector 282 and the enzyme 284/tag moiety 286 entity conjugates with connector 282. Thus, the reaction can be amplified and thus the detection signal can be amplified. The enzyme or catalyst 284 activates the subject probe 288 (e.g., tyramide probe) and, once activated, activated subject probe 288′ binds to and detectably labels neighbor molecule 211 and neighbor molecule 221, biomolecules in its vicinity. Once neighbor molecule 211 and neighbor molecule 221 are labeled, cell 108a can be further analyzed, such as by extraction and MS spectrometry.

FIG. 2C also shows a second, alternative labeling pathway in panels (v) and (vi). A target biomolecule 301 is processed as described above for the first pathway as shown in panels (i) and (ii). To the complex illustrated in FIG. 2C's panel (ii), an enzyme-bearing reactant 207 (also referred to interchangeably herein as enzyme-bearing reactive molecule unless specific context indicates otherwise) is added. Enzyme-bearing reactant 207 reacts with the activated probe 205a conjugated to the target biomolecule 301. In this example, the enzyme-bearing reactant 207 includes a reactive moiety 271 and an enzymatic moiety 273, such as maleimide-HRP or the like. FIG. 2C's panel (v) illustrates reacting the reactive moiety 271 (e.g., thiol-reactive moiety) of the enzyme-bearing reactant 207 with a free functional group (e.g., thiol group) of the activated probe 205a to form an enzyme-bearing probe 205b2 conjugated to the target biomolecule 301. The enzymatic moiety 273 may be peroxidase or ligase and activates the subject probe 288 (e.g., tyramide probe) and, once activated, activated subject probe 288′ binds to and detectably labels neighbor molecule 211 and neighbor molecule 221, biomolecules in its vicinity. Once neighbor molecule 211 and neighbor molecule 221 are labeled, cell 108a can be further analyzed, such as by extraction and MS spectrometry.

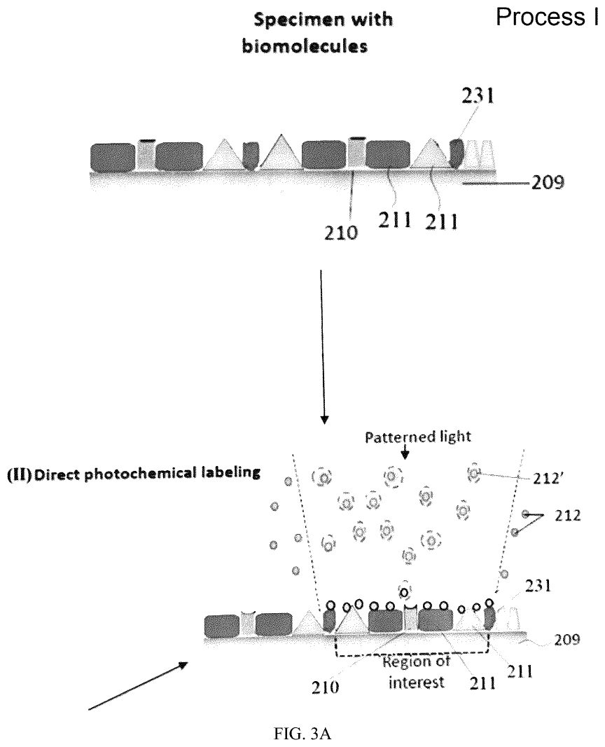

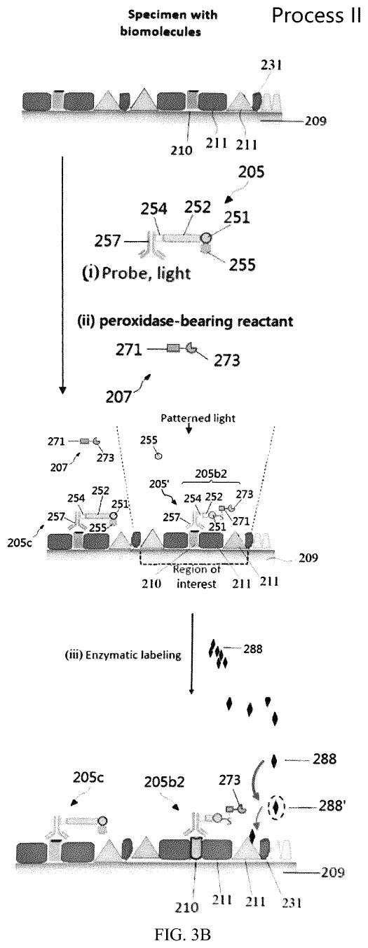

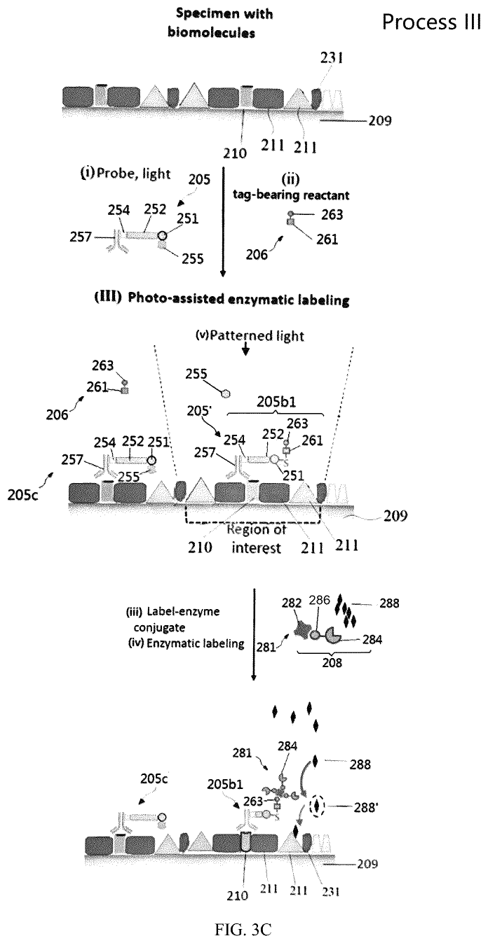

FIG. 3A shows a comparison of direct photochemical labeling (labeled Process I) and two photo-assisted enzymatic labeling (labeled Process II and Process III) using the photosensitive probes and systems described herein on a specimen with biomolecules to label biomolecules in small region of interest (ROI). Prior to performing either Process I or Process II/III, a sample (e.g., a cell or tissue sample) containing a biomolecule of interest 210 (protein will be used herein by way of example, but other biomolecules could instead be analyzed) is analyzed and a region of interest is identified. The sample can be pretreated, such as fixed and stained. For example, a sample can be fixed and stained with a cell stain (e.g., hemotoxylin and eosin (H &E); Masson's trichrome stain), identified with an immunofluorescent labeled antibody recognizing a protein of interest or by other methods. Once the region of interest is identified, a complex of neighboring biomolecules within the region of interest is analyzed. As illustrated in Process I, the sample on substrate 209 is treated with a direct photoreactive probe 212 and patterned light is directed to the sample. The patterned light activates direct photoreactive probe 212 in its pathway to form activated direct photoreactive probe 212′. The activated direct photoreactive probe 212′ is able to form complexes with other molecules within a close vicinity (show by the dotted line in Process I). The activated direct photoreactive probe 212′ diffuses and labels neighbor molecules 211 near the biomolecule of interest 210. However, the labeling diameter (300-600 nm) of photoactivation of the direct photoreactive probes is spatially restricted by the diffraction limit of the light sources used. Additionally, since the photoreactive probe is free to diffuse, any proteins in the pathway of the patterned light can be labeled. Process I also shows that it labels more distant biomolecules 231. The region labeled by activated direct photoreactive probe 212′, or labeled precision, covers a region of about 300-600 nm. This region can include biomolecules that are not in sufficiently close proximity to the protein of interest for some applications.

In contrast, in Process II illustrated in FIG. 3B and Process III illustrated in FIG. 3C, the photosensitive probe 205 is preconjugated with a bait molecule (see FIG. 2C's panel (i)) configured to recognize biomolecule of interest 210. The photosensitive probe 205 and the enzyme-bearing reactant 207 (or the tag-bearing reactant 206) are delivered to the sample on substrate 209. The enzyme-bearing reactant 207 includes a reactive moiety 271 and a enzymatic moiety 273. The tag-bearing reactant 206 includes reactive moiety 261 and tag moiety 263. As illustrated in FIG. 3C's Step (v), patterned light is also directed to the sample. However, here, (and unlike FIG. 3A Process I), patterned light uncages and activates the attached photosensitive probe 205 to allow the liberated functional group (e.g., thiol group) of the uncaged probe 205′ (probe 205′ is best seen in FIG. 2C's panel (ii)) to react with the tag-bearing reactant 206 (delivered to the sample in Step (ii) of Process Illillustrated in FIG. 3C) or the enzyme-bearing reactant 207 (delivered to the sample in Step (ii) of Process II illustrated in FIG. 3C) to thus form the tag-bearing probe 205b1 (see FIG. 3C) or the enzyme-bearing probe 205b2 (see FIG. 3B) in the selected region of interest. Steps (i) and (ii) in Process II and III also show how background or unwanted labeling is reduced using the probes and methods described herein. FIG. 3B shows in Process II Step (i) and FIG. 3C shows in Process III Step (i), a photosensitive probe 205c is attached to a biomolecule; however, since the photosensitive probe 205c is outside the light delivery region, the photosensitive probe 205c is not activated and its triggerable portion 251 remains masked by the caging group 255. Thus, there is no reaction between the photosensitive probe 205c and the enzyme-bearing reactant 207 (see FIG. 3B) or the tag-bearing reactant 206 (see FIG. 3C).

Unreacted tag-bearing reactant 206 or enzyme-bearing reactant 207 is washed away. FIG. 3B's Step (iii) in Process (III) illustrates that the enzymatic moiety 273 of the enzyme-bearing probe 205b2 attached to molecule of interest 210 activates the subject probe 288 to activated subject probe 288′, resulting in labeling of the neighbor molecules 211. Likewise, FIG. 3C's Steps (iii) and (iv) in Process III show labeling of the molecules near the molecule of interest 210 using the labeling system 208. Other labeling systems can also be used. As seen in this illustration, the labeling complex 281 conjugates with the tag moiety 263 of the tag-bearing reactant 206, and the enzyme or catalyst 284 (e.g., peroxidase) activates the subject probe 288 to generate activated subject probe 288′. Since the tag-bearing probe 205b1 is attached to molecule of interest 210, the neighbor molecules 211 are labeled, while the more distant molecule 231 is not. In addition, relative to the process II of FIG. 3B, the process III of FIG. 3C further includes a signal amplification step. as shown in FIG. 3C, the labeling system 208 includes a labeling complex 281 with a connector 282 and an enzyme or catalyst 284, and a subject probe 288. In some embodiments, the connector 282 can be avidin-dye conjugate, streptavidin-dye conjugate, neutravidin-dye conjugate or the like, and the enzyme or catalyst 284 can be tag-peroxidase and utilizes peroxide (not shown) for activity. The tag moiety 263 of the tag-bearing reactant 206 and the connector 282 recognize one another and conjugate. The tag moiety 286 of the enzyme 284/tag moiety 286 entity also recognizes connector 282 and the enzyme 284/tag moiety 286 entity conjugates with connector 282. Thus, the reaction can be amplified and thus the detection signal can be amplified. Although Processes II and III use the photosensitive probe shown in FIG. 2A to illustrate the photo-assisted enzymatic labeling, it will be appreciated that the photosensitive probe shown in FIG. 2B and related process is also applicable for FIG. 3B's Processes II and FIG. 3C's Processes III. Alternatively, relative to the photosensitive probe 205 shown in FIG. 2A, the photosensitive probe 215 shown in FIG. 2B has a higher number of units (e.g., 2 units) comprising a triggerable portion 251, a linker 252, and a caging group 255, the photoreactive probe 205 is activated by the process of photo-uncaging, leading to removal of caging group 255 from photoreactive probe 205 and leaving behind on activated photoreactive probe multiple reactive entity (e.g., two —SH entity, in this case) which can bind to the enzyme reactant 271 or the labeling reactant 261. Thus, the reaction can be amplified and thus the detection signal can be amplified.

By photoselectively localizing the enzyme-bearing reactant 207 or the labeling complex 281, near the molecule of interest and labeling the neighbor molecules 211 in the region of interest using the tagging and labeling just described, the coupling reaction can be localized to a region as small as <100 nm. In some variations, a larger region (e.g., up to about 200 nm, up to about 300 nm, up to about 400 nm, up to about 500 nm, up to about 1 μm, up to about 2 μm, up to about 5 μm) could be labeled. Furthermore, some molecules of interest in a sample have more one region of localization and hence interact with different molecular complexes in different locations simultaneously. The photo-labeling can be used successively in more than one location. For example, after applying light as shown in FIG. 3B's Process II or FIG. 3C's Process III and tagging the neighbor molecules as indicated, the light can be selectively applied to a second (third, fourth, etc.) location in the sample and this process can be repeated as many times as desired. In addition to labeling (depositing labels) to a relatively small number of neighbor molecules in a very small area of a sample, such as due to the use of the microscope analysis to direct the light and the probes described herein, and as explained below, the process can also be performed with sufficiently mild or gentle treatments so that the cell architecture remains intact.

Also disclosed herein is method of photoselectively tagging and labeling biomolecule. The method may be used to tag and/or label carbohydrates, lipids, nucleic acids, proteins, either alone or in combination. The method may include the step of treating a biological sample with a photosensitive probe having a bait molecule and a triggerable molecule with a free functional group masked or caged by a caging group and binding the bait molecule to a target molecule (i.e. a prey) in the biological sample. In some embodiments, the biological sample comprises a plurality of cells. In some embodiments, the biological sample comprises a plurality of living cells. In some embodiments, the biological sample includes at least one, at least 100, at least 1000 or at least 10,000 live cells. In some embodiments, the biological sample comprises cell extracts. In some embodiments, the triggerable molecule is coupled to the bait molecule through a chemical bond or a linker. In some embodiments, the photosensitive probe and the target molecule form a non-covalently conjugated probe-target molecule.

The methods may include the step of illuminating the biological sample with an imaging lighting source of an image-guided microscope system. The methods may include the step of imaging the illuminated sample with a camera. The methods may include the step of acquiring with the camera at least one image of subcellular morphology of the sample in a first field of view. The methods may include the step of processing the at least one image and determining a region of interest in the sample based on the processed image. The methods may include the step of obtaining coordinate information of the region of interest.

The methods may include the step of selectively illuminating with optical light the region of interest based on the obtained coordinate information to uncage the photosensitive probe and thereby to form an uncaged probe-target molecule in the region of interest. The methods may include the step of removing the unconjugated photosensitive probe from the biological sample so as to allow the photosensitive probe to guide the selectively illumination on the selected region of interest. In some embodiments, the step of selectively illuminating comprises illuminating a region for 25 μs/pixel to 400 μs/pixel, for 50 μs/pixel to 300 μs/pixel, or for 75 μs/pixel to 200 μs/pixel.

The method may include the step of delivering a tag-bearing reactive molecule or an enzyme-bearing reactive molecule to the biological sample so as to allow the tag-bearing reactive molecule or an enzyme-bearing reactive molecule to react with the uncaged probe in the selected region of interest. In some embodiments, the tag-bearing reactive molecule includes a tag moiety and a reactive moiety, while the enzyme-bearing reactive molecule includes an enzymatic moiety and a reactive moiety. In some embodiments, the tag moiety of the tag-bearing reactive molecule comprises a biotin derivative, a click chemistry tag, a HaloTag, a SNAP-tag, a CLIP-tag, digoxigenin, or a peptide tag. In some embodiments, the click chemistry tag comprises an alkyne-based or azide-based moiety. In some embodiments, the enzymatic moiety of the enzyme-bearing reactive molecule comprises a peroxidase, a ligase or the like. In some embodiments, the reactive moiety of the tag-bearing reactive molecule or the enzyme-bearing reactive molecule includes a thiol-reactive moiety. In some embodiments, the reactive moiety of the tag-bearing reactive molecule includes an iodoacetyl-based moiety, maleimide-based moiety, pyridyldithio-based moiety, or HPDP-based moiety. In some embodiments, the tag-bearing reactive molecule is an iodoacetyl-(PEG)m-biotin, maleimide-(PEG)m-biotin, pyridyldithio-biotin or HPDP-biotin, and wherein m each independently is an integer of 0-20. In some embodiments, the enzyme-bearing reactive molecule is a maleimide-HRP.

The methods may further include the step of delivering a subject probe to the sample so as to allow the enzyme-bearing reactive molecule to catalyze the subject probe (e.g. tyramide probe) to form a covalent bond between the subject probe and the biological sample. More specifically, the enzyme-bearing reactive molecule can activate the subject probe to have a free radical and form the covalent bond between the subject probe and a tyrosine of the biological sample. Alternatively, the methods may further include the step of conjugating a detectable label with the tag-bearing reactive molecule and detectably proximity labeling neighbors proximal the target molecule (i.e. the prey) with detectable label activity. In some embodiments, the detectable label comprises a catalytic label. In some embodiments, the detectably proximity labeling comprises photoselective proximity labeling a region less than 5 μm, less than 2 μm, less than 1 μm, less than 500 nm, less than 300 nm, less than 200 nm, or less than 100 nm in diameter. Some embodiments include the step of conjugating a connector with the tag-bearing reactive molecule. In some embodiments, the connector is conjugated to the tag-bearing reactive molecule through the affinity between the connector and tag-bearing reactive molecule so as to identify the location of the biological sample conjuagated with the probe. Some embodiments further include the step of conjugating a tag-enzyme to the connector. In some embodiments, the tag-enzyme is configured to catalyze a subsequently delivered subject probe (e.g. tyramide probe) to form a covalent bond between the subject probe and the biological sample. More specifically, the tag-enzyme can activate the subject probe to have a free radical and form the covalent bond between the subject probe and a tyrosine of the biological sample outside the selectively illumination region. Some embodiments further include the step of removing at least the region of interest from the microscope stage. Some embodiments further include the step of subjecting the selectively illuminated sample to mass spectrometry or sequencing analysis.

Some methods include contacting a biological sample having a target biomolecule with a photosensitive probe as described herein, washing unbound probe away, using optical radiation to spatially selectively uncage the photosensitive probe with a target biomolecule, attaching a tag or an enzyme to the biomolecule/probe complex, and selectively proximity labeling biomolecule neighbor molecules.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A photosensitive probe of formula (I): 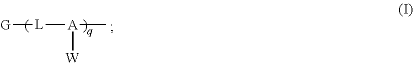
wherein
the L portion includes a chemical bond or a linker;
the G portion includes a conjugatable group for linking to a bait molecule, or a bait molecule;
the A portion includes a triggerable molecule configured to render available a functional group linking the W portion thereof upon photo-uncaging;
the W portion includes a caging group that cages the functional group of the triggerable molecule; and
q is an integer of 1-20.

2. The photosensitive probe of claim 1, wherein the probe is bound to a bait molecule;
the bait molecule is used for conjugation with a biological sample.

3. The photosensitive probe of claim 1, wherein the photo-uncaging comprises bond cleavage between the A portion and the W portion and removal of the W portion from the probe.

4. The photosensitive probe of claim 1, wherein the bait molecule is at least one of of an antibody, a CLIP-tag, a HaloTag, protein A, protein G, protein L, protein A/G, protein A/G/L, immunoglobulin binding peptides, avidin, streptavidin, neutravidin, an RNA molecule, a small molecule, a nucleic acid molecule, a fluorescent in situ hybridization (FISH) probe, fragment antigen binding region, nanobody, and a SNAP-tag.

5. The photosensitive probe of claim 4, wherein the bait molecule is a secondary antibody.

6. The photosensitive probe of claim 1, which satisfies one or more of the following conditions (1) to (5):
(1) the functional group is a thiol group;
(2) the caging group is a one- or/and two-photon sensitive caging group;
(3) the linker includes a moiety of (PEG)ₙ, peptide, amino acid, oligonucleotide or a combination thereof, and wherein n is an integer of 1-20;
(4) the conjugatable group is a click chemical group, —COOH, —NHS, a maleimide group, an iodoacetyl group or a cysteine/thiol group;
(5) the caging group is removable at a wavelength ranging from 700 nm to 1600 nm at one- or/and two-photon light source so as to render available the functional group of the triggerable molecule.

7. The photosensitive probe of claim 6, wherein the triggerable molecule is a cysteine or a derivative thereof with the thiol group thereof caged by the W portion.

8. The photosensitive probe of claim 1, wherein the caging group is a nitrodibenzofuran-based caging group or an ortho-nitrobenzyl based caging group.

9. The photosensitive probe of claim 8, wherein the caging group includes a moiety of 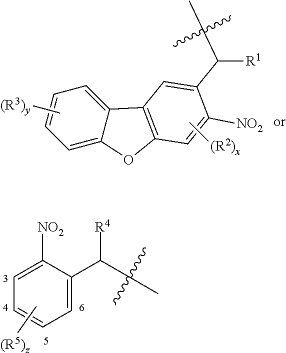
wherein
R¹ is optionally substituted alkyl, optionally substituted carbocyclyl, optionally substituted heterocyclyl, optionally substituted aryl, optionally substituted heteroaryl, —OR^{D1} or —N(R^{D1a})₂;
R² each independently is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D2} or —N(R^{D2a})₂;
R³ each independently is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D2} or —N(R^{D2a})₂;
R⁴ is H, —CO₂R^{D1}, —C(═O)R^{D1} or —C(═O)N(R^{D1a})₂;
R⁵ each independently is halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D2} or —N(R^{D2a})₂;
R^{D1} is hydrogen, optionally substituted alkyl, optionally substituted carbocyclyl, optionally substituted heterocyclyl, optionally substituted aryl, optionally substituted heteroaryl, or an oxygen protecting group;
R^{D2} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group;
R^{D1a} each independently is hydrogen, optionally substituted alkyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring;
R^{D2a} each independently is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D2a} are taken together with their intervening atoms to form a substituted or unsubstituted heterocyclic or substituted or unsubstituted heteroaryl ring;
x is 0, 1 or 2;
y is 0, 1, 2, 3, or 4; and
z is 0, 1, 2, 3, or 4.

10. The photosensitive probe of claim 9, wherein R¹ is —OH, x is 0 and y is 0; and wherein R⁴ is H, R⁵ is C₁₋₂₀ alkoxy and z is 2, with the proviso that two R⁵ are at C-4 and C-5 positions, respectively.

11. The photosensitive probe of claim 6, wherein the click chemical group is one or more of BCN, DBCO, N₃, and alkynyl.

12. The photosensitive probe of claim 6, wherein the caging group is removable at a wavelength ranging from 200 nm to 1600 nm at one- or/and two-photon light source so as to render available the functional group of the triggerable molecule.

13. A photoreactive kit, comprising:
the photosensitive probe as claimed in claim 1; and
a tag-bearing reactive molecule or an enzyme-bearing reactive molecule, having reactivity with the rendered available functional group of the triggerable molecule when it is uncaged.

14. The photoreactive kit of claim 13, wherein the tag-bearing reactive molecule includes a tag moiety and a reactive moiety; and/or
the enzyme-bearing reactive molecule includes an enzymatic moiety and a reactive moiety.

15. The photoreactive kit of claim 14, wherein the tag moiety of the tag-bearing reactive molecule is one or more of a biotin derivative, a CLIP-tag, a digoxigenin tag, a HaloTag, a peptide tag, oligonucleotide, a SNAP-tag and a click chemistry tag; and/or
the enzymatic moiety is a peroxidase or a ligase.

16. The photoreactive kit of claim 15, wherein the biotin derivative includes the moiety of 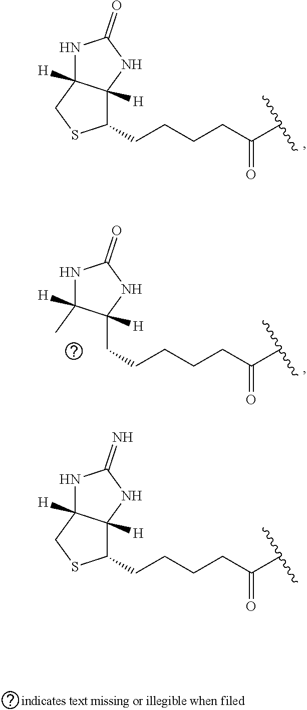
or a derivative thereof; and/or
the click chemistry tag comprises an alkyne-based or azide-based moiety.

17. The photoreactive kit of claim 15, wherein the click chemistry tag includes the moiety of 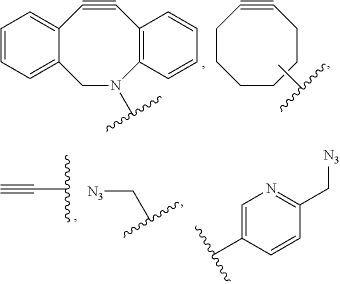
or a derivative thereof.

18. The photoreactive kit of claim 14, wherein the reactive moiety of the tag-bearing reactive molecule includes a thiol-reactive moiety; and/or
the reactive moiety of the enzyme-bearing reactive molecule includes a thiol-reactive moiety.

19. The photoreactive kit of claim 14, wherein the reactive moiety of the tag-bearing reactive molecule includes an iodoacetyl-based moiety, maleimide-based moiety, pyridyldithio-based moiety, or HPDP-based moiety.

20. A method for photoreactive labeling, the method comprising:
delivering the photosensitive probe of the photoreactive kit as claimed in claim 13 to a biological sample;
conjugating the bait molecule to a target molecule in the biological sample;
selectively illuminating a selected region of interest of the biological sample with optical radiation to uncage the photosensitive probe and thereby to generate an uncaged probe in the selected region of interest; and
delivering the tag-bearing reactive molecule or the enzyme-bearing reactive molecule of the photoreactive kit as claimed in claim 13 to the biological sample so as to allow the tag-bearing reactive molecule or the enzyme-bearing reactive molecule to react with the uncaged probe in the selected region of interest.
